(19) United States
(12) Patent Application Publication
(10) Pub. No.: US 2014/0209550 A1
(43) Pub. Date: Jul. 31, 2014

(54) LINE ORGANIZER AND MANAGEMENT SYSTEM FOR INFUSION SYSTEMS

(71) Applicants: Jeffery Pryor, Carlsbad, CA (US); William Patrick Dickinson, Carlsbad, CA (US)

(72) Inventors: Jeffery Pryor, Carlsbad, CA (US); William Patrick Dickinson, Carlsbad, CA (US)

(73) Assignee: Pryor Products

(21) Appl. No.: 13/750,644

(22) Filed: Jan. 25, 2013

(51) Int. Cl. A61M 5/14

(52) U.S. Cl. CPC A61M 5/1417
USPC 211/85.13

(57) ABSTRACT

An IV line management system providing an indicia scheme to facilitate the connection of the correct medicine to the correct pump channel and to the correct manifold inlet where the manifold is connected to a IV port on the patient. The stand can carry one or more IV pumps each with multiple channels. The stand has a hook top with indicia that may be, for example, numbers. The medicine in a particular numbered hook is connected to a pump channel which is identified by logic, the pump display, or an attached number indicator. The output channel of the pump is connected to a manifold with numbered inlets. Intermediate the pump and manifold a guide with numbered slots organizes the IV lines and releasable retains them in a numbered slot. The guide has a display surface that may be annotated with the medicine and dosage for the associated IV line

The present application is related to and claims the priority benefit of co-pending U.S. Provisional Application Nos. 61/591,101 filed Jan. 26, 2012 and 61/669,396 filed Jul. 9, 2012 both entitled Line Management Systems for Infusion System.

### BACKGROUND OF THE DISCLOSURE

Modern infusion pump (IV Pump) design has developed to the point where very precise control over the quantity of medicine to be delivered to the patient through the pump is possible. Modern IV pumps also may have warning systems to indicate when the capacity of an attached IV bags is being approached for the purposes of prompting bag replacement. Pumps also alarm whenever the pump controller detects a fault condition. However, there are a significant number of instances that despite the inherent safety of the pumps, a patient may receive an overdose of a correct medication, an under dose of the correct medication, or even a dose of an incorrect medication. This may come about because no system has been developed to manage the IV tubing (referred to herein as lines) from the IV bag, to the pump channel (there may be 3 or more channels), from the pump channel to a manifold and from the manifold to the patient. The problem is compounded when more that one pump (potentially with multiple channels each) are mounted on a wheeled equipment stand with elevated attachment structure for IV bags. The overall requirement is referred to as line management. At least one connected IV bag, pump with one or more pump channels, a patient manifold (to mix the several different medications) and, a central line port (sometimes referred to as a stick) in the patient, is referred to collectively as a fluid delivery system.

### SUMMARY OF THE INVENTION

The invention combines a pump support stand and IV fluid container support above the level of any mounted pumps, with provision for indicia associated with each IV fluid container position. The pump output is directed through channels connected to a manifold where the medicines are combined for infusion into the patient. In the exemplary embodiment the channels are IV lines. A line guide can be employed to support and separate the IV lines and to label the lines with the medication type and dosage. The features of the invention that order the lines to and from the pump are referred to herein as line organization. Line organization combined with fixed and settable indicia which identify the medicine from the source (IV bag) to the patient are referred to collectively as line management.

The fluid container support design admits of any wide variety of connectors to secure the fluid container to the IV stand, such as clamps and bolts with wing nuts and the like, referred to collectively as medicine container attachments. The exemplary embodiment uses hooks spaced along an elongated support to attach the medicine containers. The hooks and elongated support are referred to herein as the hook top and correspond to what is sometimes referred to as a “rake top”. As used herein the term medicine container is intended to encompass any shape container (rigid or flexible) which can contain liquid medication. In the exemplary embodiment IV bags are illustrated as medicine containers. An IV bag is a flexible liquid tight bag that collapses as the medication is dispensed to avoid drawing air into the fluid delivery system. This is the form of container that is described in the exemplary embodiment. Each medicine container is associated with indicia associated with the hook top, which can be coordinated with a specific pump channel, or with a gravity feed fluid line. The pump channels may be identified by indicia corresponding to the indicia on the IV bag support and in turn identified with the appropriate inlet port on a patient manifold. A line organizer may be utilized at the patient's bedside. The manifold has multiple inlets and combines multiple medications that flow to an outlet port and are connected to IV needle inserted into a patient's vein. IV needles that are left in place for an extended period are referred to as a central IV port. The line organizer incorporates retention slots and may include provision for adding notations next to the relevant line to confirm that the medicine being delivered has been associated with the correct IV bag and pump channel.

Although the invention will be described in connection with a system where the indicia are numbers or letters and numbers, and the pumps may have an adjustable indicator associated with each channel, it should be understood that the pump channel number or letter may be apparent to medical personnel without any indicia on the pump, because the channels may be arranged, for example, in a vertical column, so that, for example, the highest channel would inherently be associated with the IV bag associated with the number 1 indicia and continuing in the same manner for the other channels. Similarly the indicia could, for example, be shapes or colors instead of numbers. Normally the manifold would have a minimum number of inlet ports corresponding to the total number of pump channels from one or more pumps on the same support. The inlets would be numbered in sequence. It will be understood that the indicia may consist of other means of identification than numbering. For example, different colors may be used, or the letters of the alphabet used instead of sequential numbers. In a still further variation, the pump itself may have software that displays a channel number and even the name of the medication being infused. If desired the system of the invention can accommodate an arrangement where the displayed channel number on the pump would dictate the hook on which the bag of that medicine would be hung and the manifold indicia to which the pumps output would be connected. If multiple pumps are employed the indicia on the hook top can incorporate two indicia, the first indicia might be a letter of the alphabet, such as “A” and the first channel on the A pump numbered 1 (e.g. A1). The second indicia would be identified with a number that corresponds to the associated pump channel of the “B” pump (e.g. B3) Pumps in excess of two would be associated with subsequent letters of the alphabet.

Provision is also made for attaching an adjustable channel indicators to each channel of each pump, to supplement or confirm the channel number or letter displayed by the pump software or for use where no pump or channel number is shown by the pump.

### BRIEF DESCRIPTION OF THE DRAWINGS

FIG. 1 is a perspective view of a complete fluid delivery system incorporating a stand 1 with a single vertical pole 4 with an extension 5. The pole 4 is carried on a wheeled base 3. An IV bag support 7 (also referred to as a hook top or rake top), carries three medicine bags 8, connected by a clamp 6. A single multi-channel pump 2 is supported on pole 4 and connected to manifold. As shown diagrammatically in this view the manifold is connected to the garment of patient 30 using the clip 31. The outlet 9 of the manifold is shown diagrammatically to be connected to a patient 30 and held in association with the patient's garment of using the clip 31. The connection to the patient would be through the central port on the patient (not shown).

FIGS. 2A and 2B are perspective views and show the front and back side respectively of the hook top 7 which mounts eight hooks for supporting a plurality of IV bags (not shown). Exemplary hooks are numbered 10. The hooks are shown as being on both sides of a elongated horizontal support cover 11 which in turn is supported by the by the horizontal hook support 38 (see FIG. 5). There are vertical tabs 12, 13 14 and 15 carrying indicia (in this case numbers) which are associated with each pair of hooks. The front side of the support (FIG. 2A) displays tabs as 1, 2, 3 and 4 and the backside of the support (FIG. 2B) displays the tabs as 5, 6, 7 and 8. The elongated support cover 11 is adapted to be carried on an IV pole of a pump stand through the complementary clamp parts 12 and 16 as will be more fully explained in conjunction with the description of FIG. 5.

FIGS. 3 and 3A are perspective views of an indicia indicator 18 and shows an adjustable indicia wheel 19, which allows a selected number from 1 to 8, to be displayed in the window 21. Three indicia indicators may be attached to the IV pump 2 and associated with a pump channel 32, 34, or 36 on FIG. 1, so that for example, the indicia wheel may be set to the number 1, when the pump channel is connected to an IV bag supported from the number 1 hook as indicated on the elongated cover 11, which is elevated above the pump 2 and connected to the topmost of the 3 channels on the pump. The cover 11 has upraised indicia display tabs 12, 13, 14 and 15.

FIG. 4 is a perspective view showing a manifold with 3 numbered inlet ports to correspond with the IV bags on hook numbers 1, 2 and 3, and the respective channels on the pump. In addition to the inlet ports, fluid may also enter the manifold through inlet 28. This inlet is often used for high volume flows, and can be used with an IV bag carried on hook one of the hooks and using gravity feed to deliver fluid such as fluid for re-hydrating the patient, or other fluids where precise control of the volume of flow is not necessary.

FIG. 5 is a perspective view showing the hook support 38, elongated cover 11 and the clamp parts 12 and 16. The cover is secured to the hook support 38 by bolts (not shown) which are received in the holes 18. The clamp part 12 has a bore for receiving the top pole 25 and may be held in position by an Allen bolt 39.

FIG. 6 is a perspective view showing a multiple pump configuration with 4 pumps with three channels each. The pumps 90, 92, 94 and 96 are supported on wheeled base 100. through a primary pole 102 and above a maneuvering handle 98. The pumps are clamped to extendable poles 104 and 106, and connected to IV bags on two eight hook, hook tops 112 and 114. The hook tops are carried on the extensions 108 and 110 of the extendable poles 104 and 106. In the illustration their on eight bags 116 on hook top 112, and 4 bags 120 on hook top 114.

FIG. 7 is a perspective view showing a manifold with 12 inlet ports that can be used with the pump configuration as in FIG. 6. The manifold has a hollow body 62 and twelve inlet ports 60. The outlet port 64 would be connected to the patient and the axial inlet port 66 is especially suited for infusing fluids directly from a gravity feed IV bag where precise flow control is not required.

FIG. 8 shows a multiple pump configuration with three stacked pumps 130, 132 and 134 having two channels each. The hook top 136 has eight hooks, of which hook 136 is exemplar. Six IV bags, of which bags 140 are exemplary are being supported from the hook top 136 to supply two channels on each of the three pumps. The two channels marked 142 on pump 130 are exemplary of the connections made to pumps 132 and 134. A 12 inlet port manifold 62 is shown with 6 of the inlet ports (of which the ports 60 are exemplary) being connected to correspond to the numbered hooks and their support IV bags on the hook top 136.

FIG. 9 is a perspective view of the line management system used in association with a single multi-channel pump 152. The lines 147 connecting to the pump and the lines 157 connecting from the pump the line guide 162 (FIG. 9A) are shown diagrammatically. The hook top 142 incorporates numbered indicia sections 141 that may be annotated with the medicine type that should be carried on the associate hook 10.

FIG. 9A shows the line guide 162, with slots for IV lines. In this case 8 slots are stacked vertically but on the three topmost lines are shown to be retaining IV lines. The manifold 22 (shown in more detail in FIG. 4) is in close proximity to the patient.

FIG. 9B shows the line guide 162 which incorporates retention slots 165.

FIG. 10 shows the hook top 142 of FIG. 9 in greater detail. The indicator tabs 148 have indicia shown as the numbers 1 to 4. These same numbers are repeated on medication identification sections 141. The sections 141 have a display surface that may be annotated with the type of medicine that should be contained in the associated IV bag (see bags 143 in FIG. 9). The sections 141 can also display the dosage of that medication that should be set into the pump 152.

The hook top is supported on an IV stand, with a fixed lower pole 4 a telescoping extension 15 all carried on a wheeled base 3.

FIG. 11 shows a line number indicator that can be attached to the pump 152. The construction of the number indicator is shown in greater detail as indicator 18 in FIGS. 3 and 3A.

FIG. 12 is a numbered view of another embodiment of the line guide 179, which has line guide slots 180 numbered 1 to 4 along one edge (the left edge as oriented in this Figure).

FIG. 12A shows the reverse side of the line guide 179 which has numbered slots 181 carrying numbers 5 to 8. A clip 31 may be utilized to connect the line guide to the patients garment. Since the manifold 22 may be positioned between the line guide and patient as in FIG. 9A, the line guide serves to keep the manifold in close association with the patient and IV port on the patient (not shown).

### DETAILED DESCRIPTION OF THE DRAWINGS

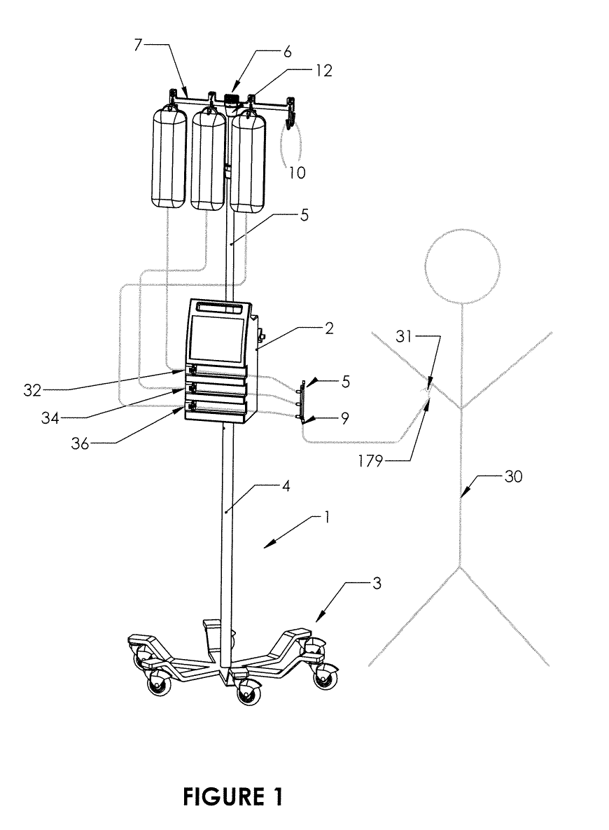

Referring to FIG. 1, there is illustrated a complete line management system. The IV Stand 1, is shown to be supporting a single IV pump which has 3 channels. The stand has a wheeled base 3, with a vertical pole 4 carrying the pump 2, an extension pole 5 extends above the pump and terminates in mount 6 for carrying the elongated support 7 which mounts 8 container supports in the exemplary form of hooks 10, with 4 hooks on each side of the elongated support 7.

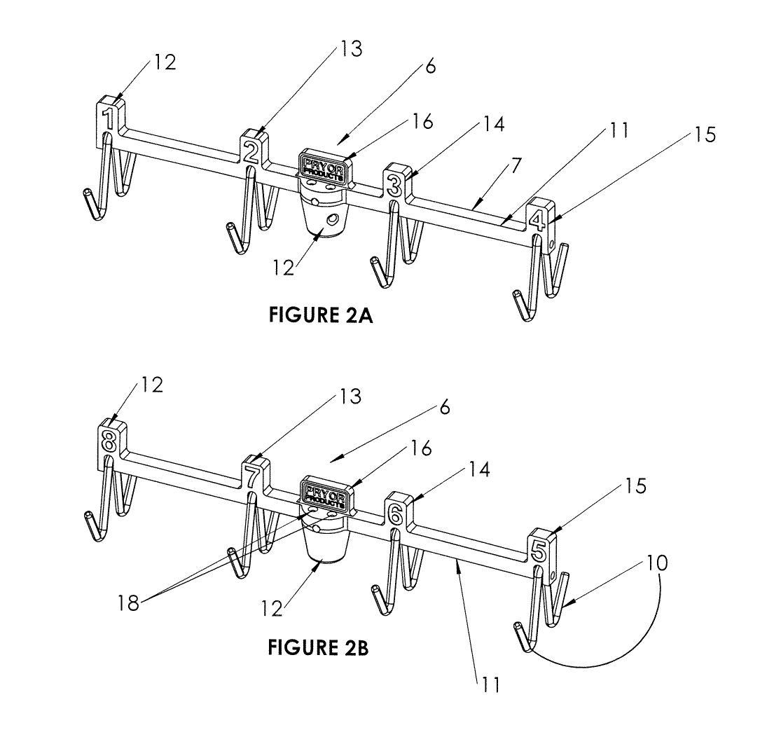

Referring to FIG. 2A, the elongated support 7, is shown to comprise a horizontal beam 11, with a plurality of upright tabs 12, 13, 14, and 15. In the exemplary embodiment, these tabs from left to right carry indicia in the form of numbers 1, 2, 3 and 4 facing the front side of the support.

The back side of the tabs is shown in FIG. 2B. The tabs are numbered 5, 6, 7 and 8. The paired hooks 10 are aligned with the numbers 1-8 and therefore an IV bag carried on the hook adjacent the numbered tab 1 would be considered the number 1 bag.

The cone shaped clamp adaptor 12 is received over the top of the pole 5 (see FIG. 1) and has a flat pad, to which the cap 16 is secured by bolts in the bolt holes 18 of which two of the four bolt holes are visible in FIGS. 2 and 2B.

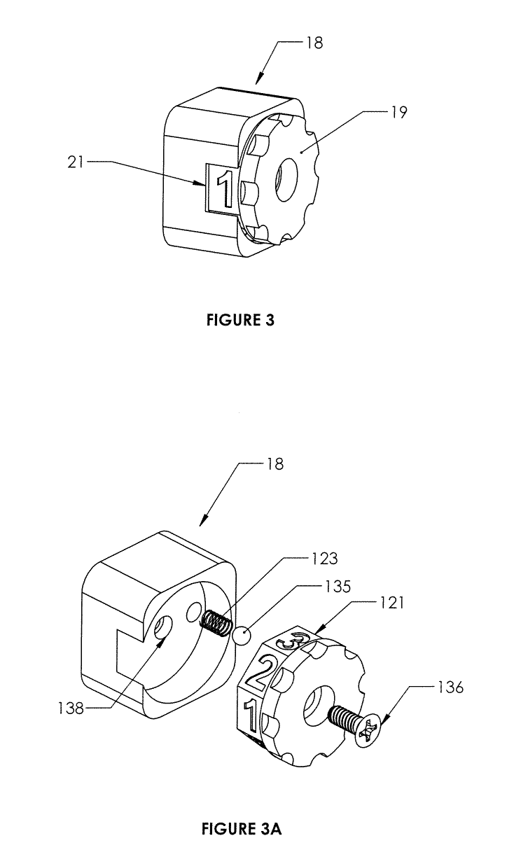

FIGS. 3 and 3A are perspective views showing an assembled indicator (Figured 3) and an exploded view (FIG. 3A). In FIG. 3, a body 18, houses a wheel 18 with an actuator knob 19 and is showing the number 1 in the window 21. Rotation of the knob allows the user to select a number for the pump channel that corresponds to the numbered hook on which the IV bag is carried. The exploded view 3A shows how the wheel 19 drives a number indicator 121. A spring 123 presses ball 135 in to recesses (not shown) in the back of the indicator wheel 121 so that the when the indicator is turned to display the next number (for example the number 2) the indicator wheel is resiliently held with the selected number centered in the window 21.

One indicator can be associated with each pump channel so that the line from the IV bag hanging on the hook associated with the number one tab would connect to the number 1 channel. For a channel where the associated indicator is set to the number 2, the indicator would be attached to pump adjacent to the second channel and the number 3 indicator would be set to 3 and attached to the pump adjacent to the third channel. As noted some pumps reference their different channels as, for example, A and B and those designations may appear on the face of the pump. In the situation where two such pumps are mounted on a pole, the indicia on the hook top would incorporate indicia 1A, 1B, 2A and 2B. Then it would only be necessary to label or associate one of the pumps as pump 1 and the other as pump 2.

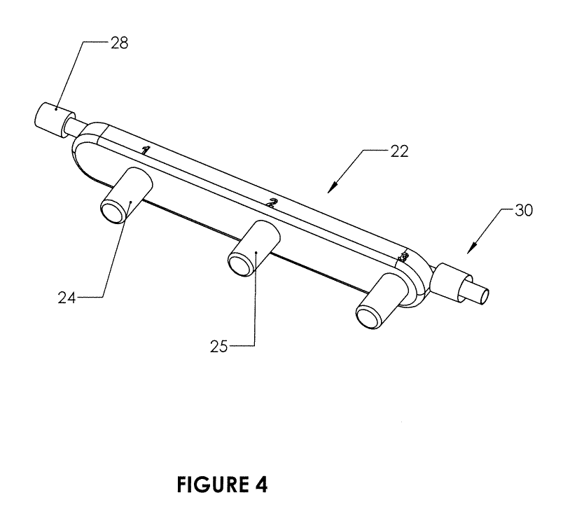

Referring to FIG. 4 there is illustrated a 3 port manifold. Each inlet port 24, 25 and 26 is numbered with the indicia 1, 2 and 3 so that, for example, the output of the pump channel 3 would be connected to the manifold inlet number 3. The outlet 28 of the manifold would be connected to the patient central IV port so that the patient would receive measured quantities of all three medicines connected to the manifold ports. The manifold also has an axial inlet 30 which provides a free flow inlet that may be especially useful with gravity feed bags for high flow uses that do not have to be precisely metered.

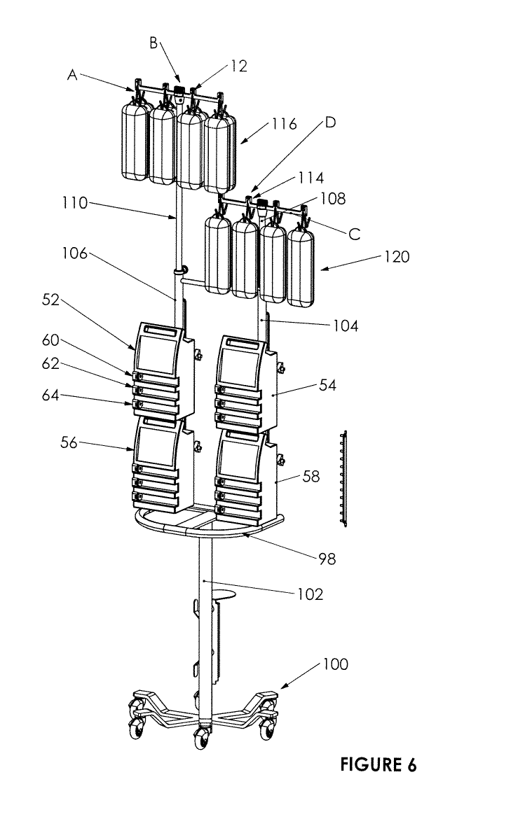

It will be understood that an IV stand may be used with multiple pumps stacked and secured to the IV pole 4, or may be arranged horizontally with an additional horizontal beam or rack secured between two vertical poles of a multiple pole stand (see FIG. 6).

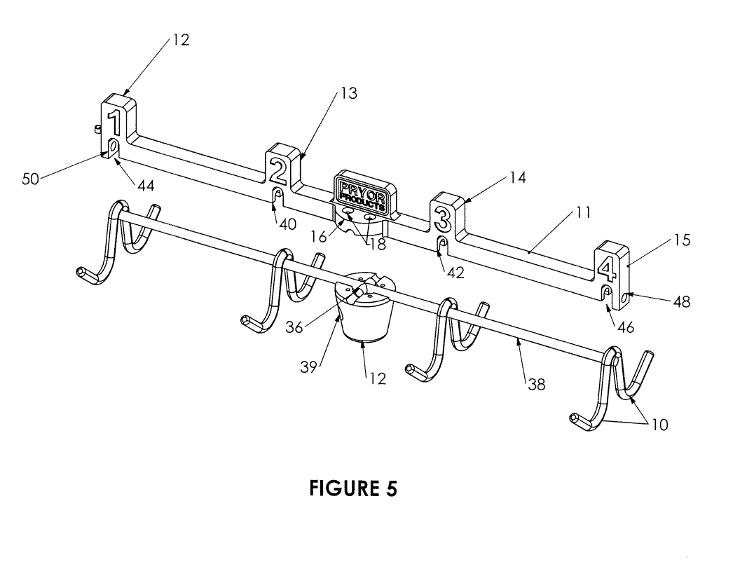

FIG. 5 is a perspective view of a hook top showing the relationship between the elongated member 11 and the support rod 32. The support rod 32 provides part of the requisite stiffness and strength to carry multiple IV bags, and also mounts 4 sets of hooks 10. Each pair of hooks are formed out of rod stock with an inverted U-shape that is secured, as be spot welding to the support rod 32. The pair of hooks terminate in an uprising portion which prevents an IV bag from becoming inadvertently detached from the hook on which the bag is supported. A right angularly related rod 36, fits within a complementary channel in the cone shaped adapter 12. When the elongated member 11 is secured to the adapter through holes 18, the rod 32 and through it the elongated member 11 are prevented from rotation relative to the adapter 12.

FIG. 6 shows a stand with 4 pumps. Pumps 52 and 54 are mounted left to right on the top row and pumps 56 and 58 are mounted left to right on the bottom row. Each pump is shown to have 3 channels such as exemplary channels 60, 62 and 64.

In one example, the first pump would normally be the upper left pump would be connected to the hook top manufactured or marked with numbers A1, A2, and A3. The second pump 54 would be labeled B and the three channels would be connected to the IV bags on the hook top labeled B1, B2 and B3. Similarly the bottom left pump would be labeled C and the channels connected to C1, C2, and C3, and finally the forth pump would be labeled D and the channels connected to the hook tops labeled D1, D2 and D3.

In the use of the line management system of FIG. 6 when used with 4 pumps which each channel associated with an indicator like that in Figure the, the indicia would be simply numbers 1-12. The 12 channels on the pumps (3 channels times 4 pumps) The indicia indicators would be mounted adjacent to each channel and adjusted to indicate channels on two pumps as 1-12.

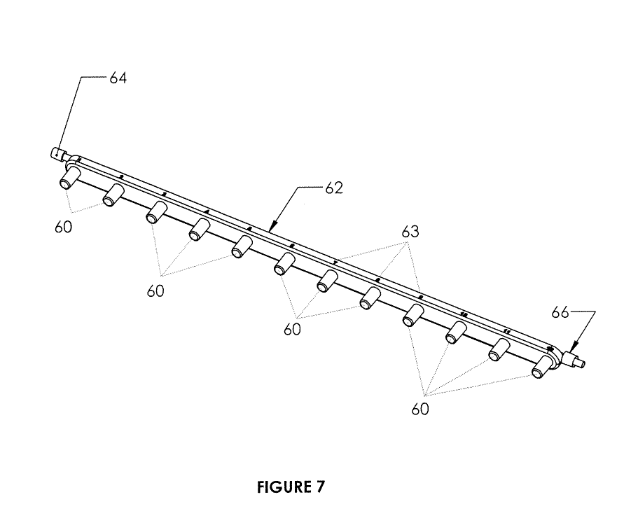

Referring to FIG. 7 and using this last discussed configuration as an example, the 12 inlet ports channels 60 carry numbers 1 through 12. These ports feed into a gallery 62. The combined fluid from ports 1-12 exits the gallery at outlet 64 and can be connected by a single line to the patient's central IV site.

A secondary inlet port 66 can be used for flow directly from an IV bag where precise control of the flow is not required.

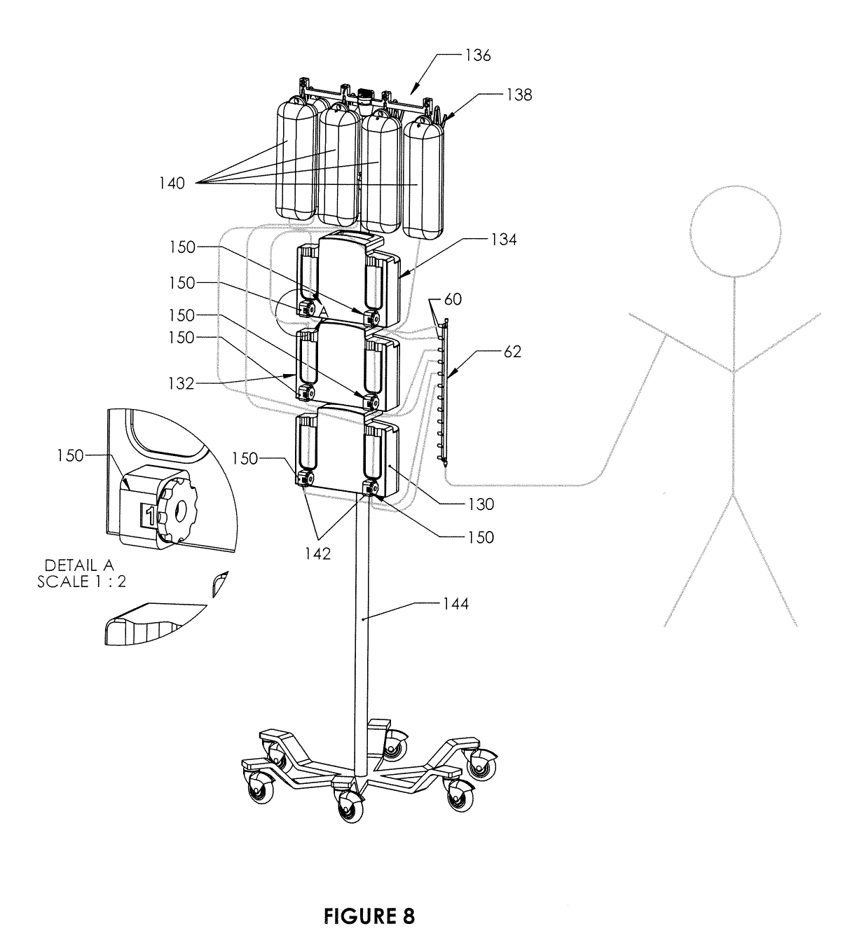

FIG. 8 is a perspective view of a 3 pump configuration vertically stacked on a single IV pole 144. Three pumps 130, 132 and 134 are mounted in vertical relationship on the pole. An 8 hook hook-top 136 is shown, and 6 IV bags 140 are supported on hooks (such as exemplary hook 13) that are numbered 1-6 (leaving hooks numbered 7 and 8 unused). The pumps illustrated each have two channels such as the exemplary channels 142. There is an indicator wheel 18 mounted adjacent each channel (exemplary indicator wheels are numbered 18 on the bottom pump). The bag on hook number 1 would be connected to the pump channel numbered 1 and the connection of the remaining 5 bags would proceed in the same manner. The output of the six pump channels are shown connected to a 12 port manifold like that illustrated in FIG. 7. Only the ports numbered 1-6 would be occupied so that the numbering would be consistent throughout.

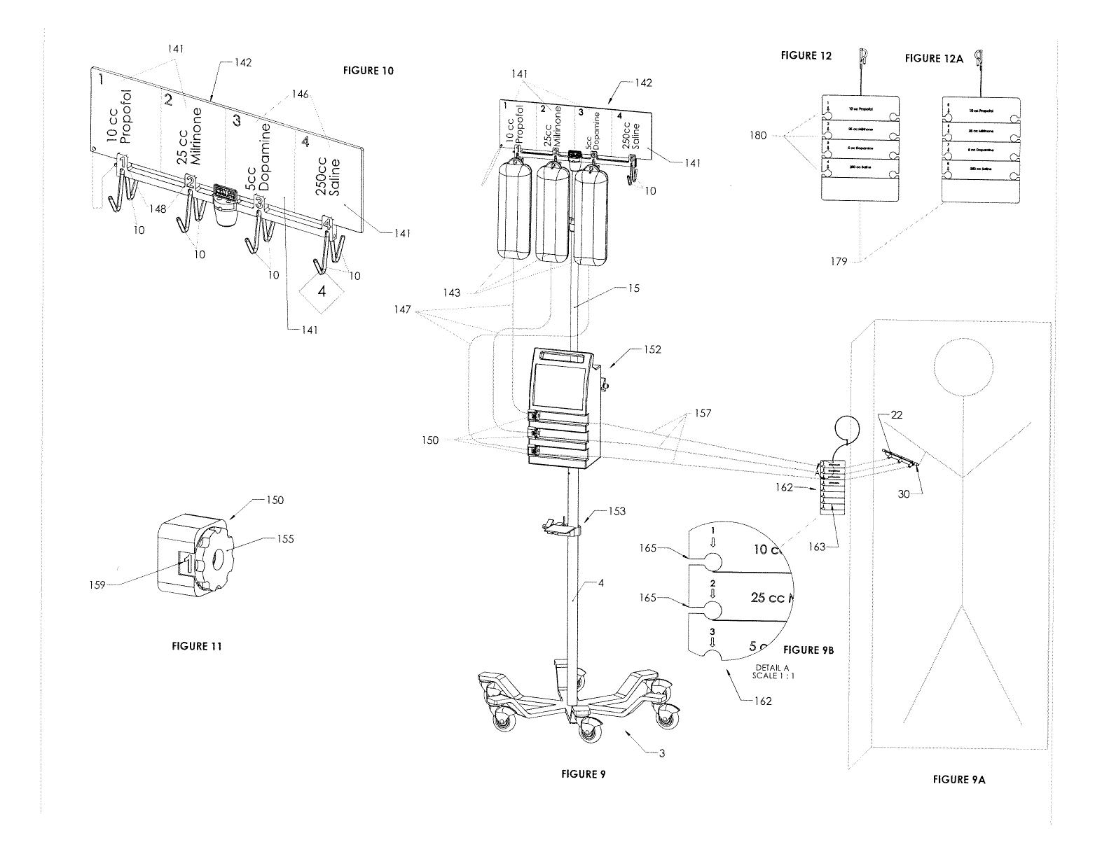

FIG. 9 shows the line management system of FIG. 1 used with a line guide 162 intermediate the pump 152 and the manifold 5. The line guide (see FIG. 10A) keeps the IV lines organized and separated and incorporates a labeling surface that can be written on with a “dry” marker. This labeling can be readily changed as medications are changed by wiping the surface with rubbing alcohol which is readily available in a hospital or clinic environment. The line guide 162 is shown with 8 openings for IV lines as shown by the exemplary openings 165 in FIG. 9B. The top three openings in FIG. 9A are shown to be occupied by lines 157 from the three channels of pump 152.

Referring again to FIG. 9, the medication identification panel 142 is illustrated. The panel is divided into 8 sections 141 of which the 4 sections 141 are visible in the front facing view in FIG. 9 and four additional sections are provided on the rear facing surface of panel 142. Each hook 10 and therefore the associated IV bag, such as bags 143 supported on respective hooks is associated with one of the 8 panel sections 141. The medicine type and dosage may be written on the respective section by a dry marker or may be appended by paper sheets from the sheet holder 153. The sheet may be provided with reinforce openings to allow them to be hung on the hooks, or may incorporate releasable adhesive so that they may be placed directly on the sections 141.

FIG. 10 is and enlarged view of the panel 142, showing exemplary medication notations on the sections 141. In this configuration the vertical tabs 148 have a slot (not shown) to receive the panel 142. The panel 142 may be lifted out of the slot to allow the notations on the sections 141 to be made on a convenient writing surface.

FIG. 11 shows a number indicator 150 with a knurled knob 155 which can be rotated to display a selected number in the window 159. This number indicator corresponds in structure and function to that shown in FIGS. 3 and 3A A number indicator 150 can be attached to the pump 152 in association with each pump channel, so that the indicia can be made consistent throughout the line management system. For example if numbers are to be used, the hook number 1 identified by the panel section 142 would be connected to the first channel of the pump and the number indicator 150 set to display the number 1. the output line of that pump channel can be secured in the opening 165 labeled one (see FIG. 9B). then the line would be connected to the patient manifold inlet 24 which is labeled 1 (see FIG. 4). Consistent indicia through all the components of the system makes more intuitive the task of insuring the right medication in the right dose is delivered to the patient. In a busy hospital environment this approach simplifies the task for the nurse or medical technician and reduces the potential for error. Of course proper training and supervision in using the system is essential. Once learned the potential for errors is reduced and makes any errors more quickly apparent.

As will be apparent from the examples given the line management system of the invention provides for an extremely flexible system that accommodates all know IV pumps with no modification and the addition only of an easily attached indicia wheel and provides a consistent and intuitive process for attaching lines from the IV bags, to the pump channels and to the manifold. The fact that the system is simple and intuitive should reduce the frequency of errors that have resulted in the use of the equipment without a line management system.

The following claims are meant to show the intended scope of the invention and to serve as a Statement of the Invention.

1. A line management system for medicine delivery to a patient, comprising:
a support for carrying at least one IV pump;
said support having an elongated support member adapted to carry a plurality of medicine container attachments,
a plurality of medicine delivery channels in hydraulic communication with medicine containers supported on said elongated support member,
said elongated member having provision for mounting indicia which provide a unique identifier for each of said plurality of medicine container attachments.

2. A line management system for medicine delivery to a patient, comprising:
a supporting stand,
said supporting stand having an elongated support member mounting a plurality of medicine container attachments,
said support member having a plurality of indicia mounts spaced to substantially correspond to the location of said medicine container attachments,
indicia associated with each indicia mount providing a unique identifier for each of said plurality of medicine container attachments,
medicine delivery conduits for hydraulic communication with at least one IV pump mounted on said stand, and
medicine delivery conduits extending from said pump mounted on said stand to a manifold for mixing the medications and delivering the mixed medications to the patient.

3. A line management system for medicine delivery to a patient, according to claim 1, wherein:
said stand incorporating at least one substantially vertical support pole,
the upper end of said support pole having at least one mounting pad,
an elongated member having provision for carrying a plurality of medicine containers and adapted for being connected to said support pole.

4. A line management system for medicine delivery to a patient, according to claim 3, wherein:
a support cover received over and substantially co-extensive with said elongated member said support cover has raised indicia tabs which are adapted to display indicia corresponding to an IV container carried below each said indicia tab.

5. A line management system for medicine delivery to a patient, according to claim 4, wherein:
said indicia tabs have integral indicia displays.

6. The line management system for medicine delivery to a patient, according to claim 4, wherein:
said elongated member and said support cover are secured to said mounting pad.

7. The line management system of claim 1, wherein:
said elongated member is substantially horizontal.

8. The line management system of claim 1, wherein:
said IV pump has a plurality of channels for pumping different doses of different medicines

9. The line management system of claim 3, wherein:
an indicia indicator for being mounted on said IV pump in proximity to a particular channel and being settable so that the indicia associated with said medicine container and settable so that the indicia associated with an IV container is the same as the indicia associated with said particular pump channel.

10. The line management system of claim 1, wherein:
said medicine containers comprise IV bags,
said medicine container attachments comprise a plurality of hooks positioned along said elongated member for being received in openings in said IV bags.

11. The line management system of claim 10, wherein:
said plurality of hooks is positioned along the front and back of said elongated member.

12. The line management system of claim 2, wherein:
said medicine deliver conduits comprise IV lines,
a guide having a plurality of slots sized to receive and retain said IV lines is positioned between said pump and said manifold.

13. The line management system of claim 12, wherein:
said guide incorporates an erasable writing surface next to each slot.

14. The line management system of claim 12, wherein:
said slots are identified with indicia corresponding to the indicia associated with said medicine containers.

15. A line management system for medicine delivery to a patient, according to claim 2, wherein:
said stand incorporating at least one substantially vertical support pole,
the upper end of said support pole having at least one mounting pad,
an elongated member having provision for carrying a plurality of medicine containers and adapted for being connected to said support pole.

16. A line management system for medicine delivery to a patient, according to claim 3, wherein:
a support cover received over and substantially co-extensive with said elongated member,
said support cover has raised indicia tabs which are adapted to display indicia corresponding to an IV container carried below each said indicia tab.

17. A line management system for medicine delivery to a patient, according to claim 16, wherein:
said indicia tabs have integral indicia displays.

18. The line management system for medicine delivery to a patient, according to claim 16, wherein:
said elongated member and said support cover are secured to said mounting pad.

19. The line management system of claim 3, wherein:
said elongated member is substantially horizontal.

20. The line management system of claim 2, wherein:
said IV pump has a plurality of channels for pumping different doses of different medicines.

21. The line management system of claim 2, wherein:
indicia indicators for being mounted on said IV pump in proximity to each pump channel and being settable so that the indicia associated with the same indicia associated with the corresponding one of said medicine containers.

22. The line management system of claim 2, wherein:
said medicine containers comprise IV bags,
said medicine container attachments comprise a plurality of hooks positioned along said elongated member for being received in openings in said medicine containers.

23. The line management system of claim 10, wherein:
said plurality of hooks is positioned along the front and back of said elongated member.

24. The line management system of claim 2, wherein:
said medicine deliver conduits comprise IV lines,
a guide having a plurality of slots sized to receive and retain said IV lines is positioned between said pump and said manifold.

25. The line management system of claim 24, wherein:
said guide incorporates an erasable writing surface next to each said slot.

26. The line management system of claim 25, wherein:
said slots are identified with indicia corresponding to the indicia associated with said medicine containers.

27. The line management system of claim 7, wherein:
a medication identification panel substantially co-extensive with said elongated member and having an erasable surface on which medication details can be annotated.

28. The line management system of claim 27, wherein:
said medication identification panel is divided into sections where each such section is positioned directly above each medicine container attachment.

29. The line management system of claim 2, wherein:
said manifold has a plurality of medicine inlets for being connected to a corresponding medicine delivery conduit, and
said medicine inlets are associated with indicia which correspond to the indicia on the corresponding medicine container attachments on said elongated member.
